(19) United States
(12) Patent Application Publication
(10) Pub. No.: US 2005/0026871 A1
(43) Pub. Date: Feb. 3, 2005

(54) Method of increasing bioavailability of alendronate or other bis-phosphonate by predose administration of vitamin D derivative

(76) Inventors: Moshe Flashner-Barak, Petach Tikva (IL); E. Itzhak Lerner, Petach Tikva (IL); Vered Rosenberger, Jerusalem (IL)

Correspondence Address: KENYON & KENYON, One Broadway, New York, NY, 10004-1050, US

(21) Appl. No.: 10/738,035

(22) Filed: Dec. 16, 2003

Related U.S. Application Data
(63) Continuation-in-part of application No. 10/196,766, filed on Jul. 17, 2002.
(60) Provisional application No. 60/433,685, filed on Dec. 16, 2002.
(60) Provisional application No. 60/460,206, filed on Apr. 2, 2003.

(51) Int. Cl. A61K 31/675; A61K 31/66; A61K 31/59

(52) U.S. Cl. 514/102; 514/167; 514/89

(57) ABSTRACT

The present invention relates to a method of increasing the bioavailability of a bis-phosphonate such as alendronate by administering an effective predose of a vitamin D derivative at least 6 hours before administering a therapeutic dose of the bis-phosphonate.

This application is a continuation-in-part of U.S. Ser. No. 10/196,766 filed Jul. 17, 2002. This application claims the benefit of U.S. Provisional Patent Applications Ser. Nos. 60/433,685, filed Dec. 16, 2002 and 60/460,206, filed Apr. 2, 2003, both of which are incorporated in their entirety herein.

### FIELD OF THE INVENTION

The present invention relates to a method of increasing the bioavailability of a bis-phosphonate by administering an effective predose of a vitamin D derivative that stimulates calcium absorption, and then administering a therapeutic dose of a bis-phosphonate such as alendronate.

### BACKGROUND OF THE INVENTION

Treatment of osteoporosis, metastatic bone disease, and Paget's disease can benefit from improvements in controlled gastric release and multiple dose delivery technology. Bis-phosphonates such as alendronate, risedronate, etidronate, zoledronate, and tiludronate are commonly prescribed drugs for treatment of these diseases. Despite their benefits, bis-phosphonates are reported to have very poor oral bioavailability. Alendronate reportedly has less than 1% bioavailability. Gert, B. J., Holland, S. D., Kline, W. F., Matuszewski, B. K., Freeman, A., Quan, H., Lasseter, K. C., Mucklow, J. C., Porras, A. G. “Studies of The Oral Bioavailability of Alendronate,” Clinical Pharmacology & Therapeutics 1995, 58, 288-298. Its absorption is inhibited by foods and beverages other than water. Id. Reported side effects experienced by patients who have taken alendronate include irritation of the upper gastrointestinal mucosa. Liberman, U. A., Hirsch, L. J.; “Esophagitis and Alendronate” N. Engl. J. Med., 1996, 335, 1069-70. This irritation can lead to more serious conditions. Physicians' Desk Reference, Fosamax, Warnings. According to the literature, alendronate is best absorbed from the upper GI tract (duodenum and jejunum). Lin, J. H. “Bisphosphonates: A Review of Their Pharmacokinetic Properties,” Bone, 1996, 18, 75-85; Porras, A. G., Holland, S. D., Gertz, B. J., “Pharmacokinetics of Alendronate,” Clin. Pharmacokinet 1999, 36, 315-328. Studies show that alendronate is best absorbed at a pH of ˜6. Gert, B. J., Holland, S. D., Kline, W. F., Matuszewski, B. K., Freeman, A., Quan, H., Lasseter, K. C., Mucklow, J. C., Porras, A. G. “Studies of The Oral Bioavailablity of Alendronate,” Clinical Pharmacology & Therapeutics, 1995, 58, 288-298. As discussed in commonly-assigned U.S. Pat. No. 6,476,006, controlled gastric release of alendronate would allow for extended delivery of the drug to the duodenum and jejunum parts of the intestine and should result in improved bioavailability, and thus allow lower dosing and less irritation.

Over the last thirty years, calcium supplementation, along with vitamin D or vitamin D derivatives such as calcitriol, have been used for treating the problems of osteoporosis. Cannigia, A., Vattimo, A. “Effects of 1,25 Dihydroxycholecalciferol on Calcium Absorption in Postmenopausal Osteoporosis,” Clin. Endocrinol., 1979, 11, 99; Riggs, B. L., Nelson, K. L. “Effect of Long Term Treatment with Calcitriol on Calcium Absorption and Mineral Metabolism in Postmenopausal Osteoporosis, J. Clin. Endocrinol. Metab. 1985, 61, 457; Reid, I. R., Ames, R. W., Evans, M. C., Gamble, G. D., Sharpe, S. J. “Long Term Effects of Calcium Supplementation on Bone Loss and Fracture in Post-menopausal Women, a Randomized Controlled Trial, Am. J. Med., 1995, 98, 331. Calcitriol (1,25-dihydroxyvitamin D₃) is a vitamin D derivative that is active in the regulation of the absorption of calcium from the gastrointestinal tract. Physicians' Desk Reference, Rocaltrol Oral Solution, Description. Calcitriol is the biologically active form of vitamin D₃ and stimulates intestinal calcium transport. Merck Index, 13th Ed., 1643. References report that calcitriol is rapidly absorbed from the intestine and reaches peak serum concentrations within three to six hours after ingestion. Physicians' Desk Reference, Rocaltrol Oral Solution, Pharmacokinetics. Calcitriol is used to treat calcium deficiency.

Over the past several years, trials have been performed that indicate that there is a synergistic effect in using a combined therapy of calcitriol and bis-phosphonates. Frediani, B., Allegri, A., Bisogno, S., Marcolongo, R. “Effects of Combined Treatment with Calcitriol Plus Alendronate on Bone Mass and Bone Turnover in Postmenopausal Osteoporosis-Two Years of Continuous Treatment,” Clin. Drug Invest. 1998, 15, 223; Masud, T., Mulcahy, B., Thompson, A. V., Donnolly, S., Keen, R. W., Doyle, D. V., Spector, T. D., “Effects of Cyclical Etidronate Combined with Calcitriol Versus Cyclical Etidronate Alone on Spine and Femoral Neck Bone Mineral Density in Postmenopausal Women,” Ann. Rheum. Dis., 1998, 57, 346; Malvolta, M., Zanardi, M., Veronesi, M., Ripamonti C., Gnudi, S. “Calcitriol and Alendronate Combination Treatment in Menopausal Women with Low Bone Mass,” Int. J. Tissue React. 1999, 21, 51; Nuti, R., Martini, G., Giovani, S., Valenti, R. “Effect of Treatment with Calcitriol Combined with Low-dosage Alendronate in Involutional Osteoporosis,” Clin. Drug Invest., 2000, 19, 56. The goal of the combined therapy is to improve therapeutic results and lower the dosage of the two drugs. In these trials the drugs were given individually. International Publication WO 2001/028564 discloses a tablet containing a combination of calcitriol and alendronate in a particular range of ratios of the two drugs.

It would be desirable in combination therapy with a bis-phosphonate and a calcium transport stimulator, to be able to release the bis-phosphonate in the patient's stomach after the vitamin D derivative has been released. However, the average residence time of a pharmaceutical tablet in the stomach is about an hour. Thus, a pharmaceutical dosage form may pass through the stomach and into the intestine before the active ingredient has been completely released, especially if the dosage form delays or sustains the release of the active ingredient. If the dosage form is retained in the stomach, however, the bis-phosphonate could be released an hour or more after the vitamin D derivative upstream of the small intestine where the bis-phosphonate is most readily absorbed.

Formulation specialists have developed methods to increase the retention time of oral dosage forms in the stomach. One of the general methods involves using an intragastric expanding dosage form that swells upon contact with stomach juices, preventing its passage through the pylorus. Some intragastric expanding dosage forms use hydrogels, which expand upon contact with water, to expand the dosage form to sufficient size to prevent its passage through the pylorus. An example of such a dosage form is described in U.S. Pat. No. 4,434,153.

As reviewed by Hwang, S. et al. “Gastric Retentive Drug-Delivery Systems,” Critical Reviews in Therapeutic Drug Carrier Systems, 1998, 15, 243-284, one of the major problems with intragastric expanding hydrogels is that it can take several hours for the hydrogel to become fully hydrated and to swell to sufficient size to obstruct passage through the pylorus. Since food remains in the stomach on average from about 1 to 3 hours, there is a high probability that known expanding dosage forms like that of the '153 patent will pass through the pylorus before attaining a sufficient size to obstruct passage.

In combination with developing improved controlled release systems, those in the art are developing delivery systems that deliver multiple doses of a medication by administration of a single dose unit. An example of such a delivery system is described in U.S. Pat. No. 5,837,248. The '248 patent discloses an improved dosing of a medication whereby two or more effective, time-separated doses may be provided by administration of a single dose unit comprising two groups of particles: immediate-release particles and delayed-release particles, both containing the same active drug.

In a co-pending patent application, U.S. patent application No. 20030158154 filed Jul. 17, 2002, and U.S. Provisional Patent Application Ser. No. 60/305,913, filed Jul. 17, 2001, the current inventors disclose a method for improving the absorption of bis-phosphonates by predosing with a vitamin D derivative 2 to 6 hours before dosing the alendronate. In U.S. Provisional Patent Application Ser. No. 60/433,685 filed Dec. 16, 2002, the present inventors disclose a method for improving bis-phosphonate bioavailability by predosing with alphacalcidol 6 to 12 hours before dosing the alendronate. In U.S. Provisional Patent Application Ser. No. 60/460,206 filed Apr. 2, 2003, the present inventors disclose a method for improving the bis-phosphonate bioavailability by predosing with calcitriol in a delayed-release delivery system. The above referenced patent applications, 60/305,913; 20030158154; 60/433,685; 60/460,206, are hereby incorporated by reference in their entirety.

There remains a need for an improved controlled delivery system and an improved dosing regimen for a bis-phosphonate and a calcium transport stimulator in order to fully realize the advantages of combined therapy.

### SUMMARY OF THE INVENTION

In one aspect, the present invention provides a method of increasing the bioavailability of a bis-phosphonate comprising administering an effective predose of a vitamin D derivative, especially calcitriol, alphacalcidol, 24,25-dihydroxy vitamin D₃, and calcifediol, and after a time interval, especially about 6 hours to about 14 hours, administering a therapeutic dose of bis-phosphonate, especially alendronate, risedronate, etidronate, zoledronate, and tiludronate.

In another aspect, the time interval is about equal to the amount of time required for blood calcium level to reach a maximum after administering the vitamin D derivative. The present method especially provides for the predose of a vitamin D derivative to be administered at bedtime and the dose of a bis-phosphonate to be administered before eating. The time interval is achieved by changing the time of administration of vitamin D derivative, changing the time of administration of bis-phosphonate, and by using delay-release technology known in the pharmaceutical art.

### DETAILED DESCRIPTION OF THE INVENTION

An object of the invention is to provide dosage forms that enable improvement in combination therapy with bis-phosphonates and calcium transport stimulators like calcitriol. The improved therapy is realized with this invention by taking advantage of the fact that a calcium transport stimulator depletes the calcium concentration in the intestine, in addition to its recognized benefit of increasing calcium in the blood. Complexation of a bis-phosphonate with calcium in the gut inhibits its absorption. Thus, there is a previously unrecognized potential benefit of increasing the bioavailability of the bis-phosphonate through combined therapy. However, there is a delay of several hours between when the calcitriol enters the intestine and when the blood calcium level peaks. Maximum calcium depletion in the intestine should coincide with the peak in blood calcium level. Blood calcium concentration is measured from whole blood. Therefore, in order to release the bis-phosphonate into an environment maximally depleted of calcium, the bis-phosphonate must be retained in the stomach and its release must be delayed for several hours. Alternatively, calcitriol, or another vitamin D derivative, can be administered separately and several hours before the bis-phosphonate.

The optimum time for maximum absorption of bis-phosphonate depends on the vitamin D derivative used. The bis-phosphonate should be administered when the vitamin D derivative achieves maximum activity. Different vitamin D derivatives take different amounts of time to attain maximum activity. Thus, the time between administration of vitamin D derivative and administration of bis-phosphonate should vary according to the identity of the vitamin D derivative used. The time interval can be varied by changing the time of administration of vitamin D derivative, changing the time of administration of bis-phosphonate, and by using delay-release technology known in the pharmaceutical art.

Thus, in one embodiment, the present invention provides a method of improving the bioavailability of a bis-phosphonate, especially, alendronate, by administering a combination drug regimen that includes the steps of administering a pre-dose of a vitamin D derivative and, about 2 to about 6 hours later, administering a therapeutic dose of a bis-phosphonate. The vitamin D derivatives and bis-phosphonates useful in the practice of this and other embodiments herein described are the same. Preferably, the vitamin D derivative is calcitriol and the bis-phosphonate is alendronate.

In another embodiment, the present invention provides a method of increasing the bioavailability of a bis-phosphonate by administering an effective predose of a vitamin D derivative and, at least about 6 hours later, preferably about 6 hours to about 14 hours later, administering a therapeutic dose of a bis-phosphonate. In this embodiment, the preferred vitamin D derivative is alphacalcidol and the preferred bis-phosphonate is alendronate.

In another embodiment, the present invention provides a method of increasing the bioavailability of a bis-phosphonate by administering a delayed-release effective predose of a vitamin D derivative and, at least about 6 hours later, preferably about 6 hours to about 14 hours later, administering a therapeutic dose of a bis-phosphonate. Preferably, the release of the vitamin D derivative is delayed about 3 to about 5 hours after the vitamin D derivative is administered. In this embodiment, the preferred vitamin D derivative is calcitriol and the preferred bis-phosphonate is alendronate.

Each of these preferred embodiments includes a time interval between the administration of the effective predose of vitamin D derivative and the administration of the therapeutic dose of bis-phosphonate. The time interval can be expressed as T=t2−t1, where T is the time interval, t1 is the time at which the vitamin D derivative is administered, and t2 is the time at which the bis-phosphonate is administered. The time interval should be about equal to the amount of time required for blood calcium level to reach a maximum after administering the vitamin D derivative. By adjusting the time interval, one can release the bis-phosphonate into an environment of minimum calcium, thereby increasing the bioavailability of bis-phosphonate.

As used herein, bioavailability means “the fractional extent to which a dose of drug reaches its site of action or a biological fluid from which the drug has access to its site of action;” “the fraction of drug absorbed as such into the systemic circulation.” Goodman and Gilman's The Pharmalogical Basis of Therapeutics 5, 18 (Joel G. Hardman et. al. eds., McGraw Hill Pub. 10th ed. 2001). Oral bioavailability can be estimated based on secondary information (e.g., urinary excretion or the amount of the drug excreted unchanged in the urine, expressed as a percentage of the administered dose). Id. at 1918.

Administration of the vitamin D analog in the combination drug regiment can be by any means known in the art. Solid oral dosage forms are preferred.

Administration of the bis-phosphonate in the combination drug regimen can also be by any means known in the art. Administration via a solid oral dosage form is preferred. The solid oral dosage form can be of the conventional type well known in the art (e.g. Fosamax®), or it can be of the gastric retention type herein described.

The therapeutic or prophylactic doses of vitamin D derivative and bis-phosphonate to be administered in this combination drug regimen are the same as in other embodiments of the invention.

The dosage forms of another embodiment of the present invention enable improved combination therapy with bis-phosphonates and calcium transport stimulators by releasing the calcium transport stimulator in an immediate or uncontrolled manner, by swelling to a size that prevents passage through the pylorus and by releasing the bis-phosphonate in the stomach after a delay time period to allow the calcium transport stimulator to deplete the upper GI tract of calcium. After a delay period of preferably an hour or more, more preferably from about 2 to about 6 hours, the bis-phosphonate is released in the stomach in either an immediate or sustained release manner. Afterwards, the swollen tablet degrades or erodes into particles that are sufficiently small to traverse the pylorus.

Preferably, the pharmaceutical dosage form is retained in the stomach for about three hours or more before it breaks up, more preferably about five hours or more. In order to obstruct passage through the pylorus, the dosage form preferably swells by a factor of three or more, more preferably about eight or more, within about fifteen minutes of contacting gastric fluid. Yet more preferably, such swelling is reached within about five minutes.

Another embodiment of the present invention provides a method of increasing the bioavailability of bis-phosphonate by administering a separate predose of vitamin D derivative followed by a dose of bis-phosphonate. The predose of vitamin D derivative and the dose of bis-phosphonate can be independently administered in an immediate or in a delayed-release manner. In this embodiment, the time interval can be varied by changing the time of administration of vitamin D derivative, changing the time of administration of bis-phosphonate, and by using delay-release technology known in the pharmaceutical art.

The present invention includes the step of administering a therapeutic dose of bis-phosphonate. A therapeutic dose of bis-phosphonate is an amount of bis-phosphonate that treats or ameliorates diseases including osteoporosis, metastatic bone disease, and Paget's disease, among others.

Bis-phosphonates useful as calcium resorption inhibitors in the present invention include, for example, alendronate, risedronate, etidronate, zoledronate, and tiludronate. The most preferred bis-phosphonate is alendronate.

The dosage level of the bis-phosphonate will depend in part upon whether the dosage form is intended for delayed release or delayed/sustained release of the bis-phosphonate. A non-sustained release alendronate formulation preferably contains from about 2 mg to about 40 mg of alendronate. A delayed/sustained release alendronate formulation preferably contains from about 6 to about 120 mg of alendronate. A non-sustained release risedronate formulation preferably contains from about 20 to about 40 mg of risedronate. A delayed/sustained release risedronate formulation preferably contains from about 60 to about 120 mg of risedronate. A non-sustained release etidronate formulation preferably contains from about 200 mg to about 400 mg of etidronate. A delayed/sustained release etidronate formulation preferably contains from about 600 to about 1200 mg of etidronate. A non-sustained release tiludronate formulation preferably contains from about 200 mg to about 300 mg of tiludronate. A delayed/sustained release tiludronate formulation preferably contains from about 600 mg to about 1200 mg of tiludronate.

The bis-phosphonate can also be administered in an immediate or uncontrolled manner. An immediate or uncontrolled alendronate formulation preferably contains about 1 mg to about 100 mg, preferably about 10 mg to about 70 mg.

The bis-phosphonate may be provided in any pharmaceutically acceptable salt or acid form, salts being generally preferred because they cause less membrane irritation. Thus, alendronate includes alendronic acid and pharmaceutically acceptable salts thereof. Risedronate includes risedronic acid and pharmaceutically acceptable salts thereof. Etidronate includes etidronic acid and pharmaceutically acceptable salts thereof. Zoledronate includes zoledronic acid and pharmaceutically acceptable salts thereof. Tiludronate includes tiludronic acid and pharmaceutically acceptable salts thereof. The skilled artisan will recognize that pharmaceutically acceptable salts can exist as solvates, e.g., hydrates. One skilled in the art would recognize that these bis-phosphonates can also be provided as esters.

Alendronate is preferably provided as a monosodium salt monohydrate or trihydrate. Risedronate is preferably provided as a monosodium salt hemipentahydrate. Etidronate and tiludronate are preferably provided as hydrated or anhydrous disodium salts. Zoledronate is preferably provided as a disodium salt tetrahydrate or trisodium salt hydrate.

The present invention includes the step of administering of an effective predose of a vitamin D derivative. The skilled artisan will understand that a predose is the dose of the vitamin D derivative that is administered before the administration of the therapeutic dose of the bis-phosphonate.

Vitamin D derivatives useful as calcium transport stimulators include calcitriol, alphacalcidol, 24,25-dihydroxy vitamin D₃, and calcifediol. The most preferred calcium transport stimulator of the present invention is calcitriol. An effective predose means that the calcium transport stimulator may be dosed in any amount that results in increased intestinal absorption of the bis-phosphonate compared to an equal dose of the bis-phosphonate administered without the calcium transport stimulator. One example of an effective predose is a dose between about 0.1 μg and about 10 μg of a vitamin D derivative. A preferred dosage range is from about 0.01 μg to about 0.5 μg. A most preferred dosage is about 0.05 μg. In one embodiment, the preferred dosage of alphacalcidol is about 0.1 μg to about 10 μg, more preferably about 0.2 μg to about 2 μg. The preferred dosage of calcitriol for the embodiments employing a delayed-release predose of vitamin D derivative is about 0.1 μg to about 10 μg, more preferably about 0.2 μg to about 2 μg.

Alphacalcidol, or 1α-hydroxyvitamin D₃, is a synthetic analog of calcitriol, the hormonal form of Vitamin D₃. Alphacalcidol stimulates intestinal calcium absorption, the transport of calcium from the intestine to the bloodstream. When alphacalcidol enters the intestine, several hours must pass before blood calcium level peaks. In order to release the bis-phosphonate into an environment of minimum calcium, administration of the alphacalcidol predose should precede the administration of the bis-phosphonate dose by a time interval of several hours. A time interval of several hours, e.g. 6 hours to 14 hours, allows for maximum bioavailability of bis-phosphonate.

The maximum increase in bis-phosphonate bioavailability is observed when the time interval between administration of the alphacalcidol predose and the bis-phosphonate dose is at least about 6 hours, preferably about 6 hours to about 14 hours, more preferably about 6 hours to about 12 hours, and most preferably about 6 hours to about 10 hours. This time interval allows for a convenient dosage regimen in which the predose of alphacalcidol can be administered between 8 P.M. and midnight and the bis-phosphonate dose can be administered between 6 A.M. and 10 A.M. on the following morning. This dosing method increases the bioavailability of bis-phosphonate.

Calcitriol, or 1α,25-dihydroxyvitamin D₃, is the primary active metabolite of Vitamin D. Goodman and Gilman's The Pharmalogical Basis of Therapeutics, supra, at 1727. Like alphacalcidol, calcitriol stimulates intestinal calcium absorption. For calcitriol, blood calcium level peaks about 3 hours to about 5 hours after calctriol enters the intestine.

In a particular embodiment, the present invention provides a method of improving the bis-phosphonate bioavailability by predosing with calcitriol in a delayed-release delivery system. By delaying the release of the calcitriol predose, one can extend the optimal time between administration of calcitriol and administration of bis-phosphonate and maintain the desired time interval between administration of calcitriol and administration of bis-phosphonate. The release of the calcitriol predose is delayed about 3 hours to about 5 hours by providing the calcitriol dosage form with an enteric coating known in the art, e.g., EUDRAGIT® L, EUDRAGIT® S, cellulose acetate phthalate. Such enteric coating materials are pH-sensitive and can withstand prolonged contact with acidic gastric fluids. Therefore, the enteric coating does not dissolve until after stomach passage but dissolves readily in the mildly acidic to neutral environment of the small intestine. The level of coating necessary to achieve the desired delay of onset of drug release can be readily determined by experimentation of one skilled in the art (see, e.g., United States Pharmacopeia, 26^{th} Rev./National Formulary, 21^{st} Ed., 2002, <724> Drug Release, Delayed-Release (Enteric-Coated) Articles—General Drug Release Standard, 2160-2161; Pharmaceutical Dosage Forms and Drug Delivery Systems, H. C. Ansel, L. V. Allen, Jr., N. G. Popovich (Lippincott Williams & Wilkins, pub., 1999), Modified-Release Dosage Forms and Drug Delivery Systems, 223, 231-240).

In one embodiment using a delay-release calcitriol predose, the release of the calcitriol predose is delayed about 3 hours to about 5 hours. The time interval between delay-release calcitriol predose and bis-phosphonate dose is at least about 6 hours, preferably about 6 hours to about 14 hours, more preferably about 6 hours to about 12 hours, and most preferably about 6 hours to about 10 hours. This embodiment provides for a convenient dosage regimen in which the calcitriol predose can be administered between 8 P.M. and midnight and the bis-phosphonate dose can be administered between 6 A.M. and 10 A.M. on the following morning. This dosing method increases the bioavailability of bis-phosphonate. The convenience of this dosing method improves patient compliance.

In another embodiment, the dosage forms of this invention are retained in the stomach for an extended period of time by swelling rapidly on contact with aqueous solution, such as gastric fluid. The term “gastric fluid” means the endogenous fluid medium of the stomach, including water and secretions, or simulated gastric fluid. “Simulated gastric fluid” means any fluid that is generally recognized as providing a useful substitute for authentic gastric fluid in experiments designed to assess the chemical or biochemical behavior of substances in the stomach. One such simulated gastric fluid is USP Gastric Fluid TS, without enzymes. United States Pharmacopeia and National Formulary 24/19 p. 2235 (1999). Thus, it will be understood that throughout this disclosure and in the claims “gastric fluid” means authentic gastric fluid or simulated gastric fluid.

Rapid swelling is achieved by a gastric retention composition. The gastric retention composition may comprise a combination of a hydrogel, a superdisintegrant, and tannic acid. This composition is further described in our commonly assigned U.S. Pat. No. 6,476,006 and U.S. patent applications Ser. No. 09/887,204, hereby incorporated by reference in their entirety.

The preferred hydrogel of the gastric retention composition is hydroxypropyl methylcellulose, either alone or in combination with hydroxypropyl cellulose and/or a cross-linked acrylate polymer. Suitable cross-linked acrylate polymers include polyacrylic acid cross-linked with allyl sucrose and polyacrylic acid cross-linked with divinyl glycol. As further illustrated in the Examples, a preferred hydrogel of the invention is a mixture of hydroxypropyl methylcellulose and hydroxypropyl cellulose. The most preferred hydrogel of the present invention is a combination of hydroxypropyl methylcellulose and hydroxypropyl cellulose in a weight ratio of from about 1:3 to about 5:3. The molecular weight of the hydrogels is not critical to practice of the invention.

The gastric retention composition also may include a superdisintegrant. Superdisintegrants are pharmaceutical excipients within a larger class of excipients known as disintegrants. Disintegrants are typically hydrophilic polymers of either natural or synthetic origin. Superdisintegrants are disintegrants that swell upon contact with water. Preferred superdisintegrants of the present invention swell to at least double their non-hydrated volume on contact with water. Exemplary of these superdisintegrants are cross-linked polyvinyl pyrollidone (a.k.a. crospovidone), cross-linked carboxymethyl cellulose sodium (a.k.a. croscarmellose sodium) and sodium starch glycolate. The most preferred superdisintegrant is croscarmellose sodium.

The gastric retention composition further may include tannic acid. Tannic acid, also called tannin, gallotannin and gallotannic acid, is a naturally occurring constituent of the bark and fruit of many trees. The term “tannins” conventionally refers to two groups of compounds, “condensed tannins” and “hydrolyzable tannins.” Merck Index monograph No. 8828 (9th ed. 1976). The hydrolyzable tannins are sugars that are esterified with one or more (polyhydroxylarene) formic acids. One common polyhydroxylarene formic acid is galloyl (i.e. 3,4,5-trihydroxybenzoyl). Another common polyhydroxylarene formic acid substituent of tannins is meta-digallic acid. A common sugar moiety of tannins is glucose. The tannic acid of the present invention is selected from the hydrolyzable tannins, and especially glucose tannins in which one or more of the hydroxyl groups of glucose is esterified with gallic acid and/or meta-digallic acid. USP tannic acid is preferred for use with this invention.

The preferred gastric retention composition comprises a hydrogel, a superdisintegrant and tannic acid. These excipients more preferably are combined in a weight ratio, exclusive of the active ingredients and any other excipients that may be present, of from about 20 wt. % to about 80 wt. % hydrogel, from about 10 wt. % to about 75 wt. % superdisintegrant and from about 2 wt. % to about 15 wt. % tannic acid. A yet more preferred composition comprises from about 10 wt. % to about 35 wt. % superdisintegrant, about 5 wt. % (±2 wt. %) tannic acid, plus an amount of hydrogel sufficient to bring the total to 100 wt. %.

One especially preferred gastric retention composition comprises from about 10 wt. % to about 20 wt. % hydroxypropyl methyl cellulose, from about 45 wt. % to about 50 wt. % hydroxypropyl cellulose, from about 25 wt. % to about 35 wt. % sodium starch glycolate and from about 4 wt. % to about 6 wt. % tannic acid.

A second especially preferred gastric retention composition comprises from about 10 wt. % to about 30 wt. % hydroxypropyl methyl cellulose, from about 40 wt. % to about 60 wt. % hydroxypropyl cellulose, from about 7 wt. % to about 35 wt. % sodium croscarmellose and from about 4 wt. % to about 12 wt. % tannic acid.

The dosage form may be prepared conventionally by dry blending, dry granulation or wet granulation of the active ingredients and the gastric retention composition and any other desired excipients.

In a dry granulation, the active ingredients and excipients may be compacted into a slug or a sheet and then comminuted into compacted granules. The compacted granules may be compressed subsequently into a final dosage form. It will be appreciated that the processes of slugging or roller compaction, followed by comminution and recompression render the hydrogel, superdisintegrant, tannic acid, and active ingredients intragranular in the final dosage form. Alternatively, any of the active ingredients or excipients of the gastric retention composition may be added after comminution of the compacted composition, which results in that active ingredient or excipient being extragranular.

As an alternative to dry granulation, the blended composition may be compressed directly into the final pharmaceutical dosage form using direct compression techniques. Direct compression produces a more uniform tablet without granules. Thus, the active ingredients and any other desired excipients are blended with the composition prior to direct compression tableting. Such additional excipients that are particularly well suited to direct compression tableting include microcrystalline cellulose, spray dried lactose, dicalcium phosphate dihydrate, and colloidal silica.

An additional alternative to dry granulation is wet granulation. The blend of excipients may be granulated using an alcohol or water and alcohol mixture as a granulation solvent by standard granulation techniques known in the art followed by drying, sieving, milling and compressing into the final dosage form.

The active ingredients and gastric retention composition may be compacted using conventional compression techniques.

In a preferred dosage form of the invention, a core containing the bis-phosphonate is embedded in the gastric retention composition. Embedded tablets are an example of an embedded core type of dosage form. The dosage form may be formulated to contain the vitamin D derivative in the gastric retention composition or in a coating that is soluble in gastric fluid. A coating of the vitamin D derivative is applied over the gastric retention composition. In either formulation, the vitamin D derivative is released immediately in the stomach and will find its way to the intestine quite rapidly.

The following is an example of an immediate release bis-phosphonate core that may be used to prepare a bis-phosphonate/calcium transport stimulator dosage form of this invention. An immediate release core of bis-phosphonate may be prepared by blending the bis-phosphonate with microcrystalline cellulose, lactose, magnesium stearate and, optionally, a superdisintegrant, and compressing the blend. An exemplary formulation contains from about 20 to about 50 wt. % microcrystalline cellulose, from about 50 to about 80 wt. % lactose, from about 0.5 to about 2 wt. % magnesium stearate and from about 0 to about 5 wt. % crospovidone, sodium croscarmellose or sodium starch glycolate, plus the intended dosage of bis-phosphonate.

The following is an example of a sustained release bis-phosphonate core that may be used to prepare a bis-phosphonate/calcium transport stimulator dosage form of this invention. A sustained release core of bis-phosphonate may be prepared by blending the bis-phosphonate with hydroxypropyl methylcellulose, lactose and magnesium stearate. An exemplary formulation contains from about 5 to about 80 wt. % hydroxypropyl methylcellulose, from about 20 to about 95 wt. % lactose and from about 0.5 to about 2 wt. % magnesium stearate, plus the intended dose of bis-phosphonate.

The core may also be coated with a delayed release coating. Suitable coating substances for forming a delayed release coating include arabinogalactan; carboxymethylcellulose; gelatin; gum arabic; hydroxyethylcellulose; methylcellulose; polyvinyl alcohol; water insoluble resins such as ethyl cellulose, e.g., Ethocel™, polyamide, polymethacrylate, e.g., Eudragit™ NE, Eudragit™ RS, Eudragit™ RL, and silicones; waxes and lipids such as paraffin, carnauba wax, spermaceti, beeswax, stearic acid, stearyl alcohol and glyceryl stearates; and enteric resins such as cellulose acetate phthalate, polyvinyl acetate, hydroxypropyl methylcellulose acetate, Eudragit™ L and Eudragit™ S. The glyceryl esters may be mixed with a wax as previously described in U.S. Pat. No. 4,764,380, which is incorporated by reference in its entirety. Additional coating materials that may be used are disclosed in U.S. Pat. Nos. 4,434,153; 4,721,613; 4,853,229; 2,996,431; 3,139,383 and 4,752,470, which are hereby incorporated by reference in their entirety.

The core also may be coated with a sustained release coating to further slow release of the bis-phosphonate. Such coating materials include polymethacrylate, e.g., Eudragit™ NE, Eudragit™ RS, Eudragit™ RL, Eudragit™ L, Eudragit™ S, and mixtures of hydrophilic and hydrophobic film forming agents. Hydrophilic film forms include methyl cellulose, hydroxypropyl methylcellulose, cellulose phthalate, cellulose acetate phthalate and polyvinyl alcohol. Hydrophobic film forming agents include ethyl cellulose, cellulose acetate, hydroxypropyl methylcellulose phthalate, polyvinyl alcohol maleic anhydride copolymers, β-pinene polymers rosin, partially hydrogenated rosin and glycerol esters of rosin. A sustained release coating may be applied by methods known in the art such as by fluid bed or pan coating techniques.

The core may be embedded in the gastric composition using commercially available equipment such as a Kilian RUD-20 press coat machine.

The vitamin D derivative may be dispersed in the shell of the gastric retention composition. Thus, the vitamin D derivative may be incorporated into the preferred embedded core type dosage form by simply blending with the gastric retention composition before compression in the press coat machine.

The vitamin D derivative may be applied in a coating over the shell. The vitamin D derivative may, for example, be dissolved in ethanol with 0.1 wt. % to about 10 wt. % hydroxypropyl cellulose and then pan coated or spray coated onto the shell using coating techniques that are well known in the art.

Another preferred dosage form embodiment is a capsule. The capsule encapsulates two tablets. One tablet contains the above-described core containing the bis-phosphonate embedded in a shell of the gastric retention composition. The other tablet may be any conventional immediate release formulation containing the vitamin D derivative.

In addition to the above-described excipients, the bis-phosphonate/calcium transport stimulator dosage form may further include one or more other excipients added for any of a variety of other purposes. It will be understood by those in the art that some substances serve more than one purpose in a dosage form. For instance, some substances are binders that help hold a tablet together after compression, yet are disintegrants that help break the tablet apart once it reaches a patient's stomach. It will be further understood that the hydrogel, superdisintegrant and tannic acid of the expanding composition may serve to perform additional functions in the dosage form, which functions may already be known to those skilled in the art.

Further increase in retention times may be realized by adding a compound that produces gas when contacted with acid, such as sodium bicarbonate. Sodium bicarbonate may be provided by blending into the gastric retention composition. Sodium bicarbonate is preferably used at low concentration, of from about 0.5 wt % to about 5 wt. % of expanding composition.

Diluents increase the bulk of a solid pharmaceutical product and may make it easier for the patient and care giver to handle. Diluents include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol and talc.

Compacted dosage forms like those of the present invention may include excipients whose functions include helping to bind the active ingredient and other excipients together after compression. Binders for solid pharmaceutical compositions include, but are not limited to, acacia, alginic acid, carbomer (e.g., carbopol), carboxymethylcellulose sodium, dextrin, ethyl cellulose, gelatin, glucose, guar gum, hydrogenated vegetable oil, hydroxyethyl cellulose, hydroxypropyl cellulose (e.g., Klucel®), hydroxypropyl methylcellulose (HPMC) (e.g., Methocel®), liquid glucose, magnesium aluminum silicate, maltodextrin, methylcellulose, polymethacrylates, polyvinylpyrrolidone (e.g., Kollidon®, Plasdone®), starch, pregelatinized starch, sodium alginate and alginate derivatives.

The dissolution rate of a compacted dosage form in the patient's stomach also may be adjusted by the addition of a disintegrant or second superdistegrant to the dosage form, in addition to the superdisintegrant of the present inventive composition. Such additional disintegrants include, but are not limited to, alginic acid, carboxymethylcellulose calcium, carboxymethylcellulose sodium, colloidal silicon dioxide, croscarmellose sodium (e.g., Ac-Di-Sol®, Primellose®), crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®) and starch.

Glidants can be added to improve the flow properties of a solid composition and improve the accuracy of dosing. Excipients that may function as glidants include, but are not limited to, colloidal silicon dioxide, magnesium trisilicate, powdered cellulose, starch, talc and tribasic calcium phosphate.

When a dosage form such as a tablet is made by compaction, a composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product from the dye. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, surfactants, talc, waxes and zinc stearate.

Flavoring agents and flavor enhancers make the dosage form more palatable to the patient. Common flavoring agents and flavor enhancers for pharmaceutical products that may be included in the dosage forms of the present invention include, but are not limited to, maltol, vanillin, ethyl vanillin, menthol, citric acid, fumaric acid ethyl maltol, and tartaric acid.

The dosage forms may also be colored using any pharmaceutically acceptable colorant to improve their appearance and/or facilitate patient identification of the product and unit dosage level.

Having thus described the invention with reference to certain preferred embodiments, it is further illustrated by the following non-limiting examples.

### EXAMPLES

### Example 1

Sodium alendronate monohydrate is formulated into an extended release core of 5-mm diameter with a composition shown in Table 1 by mixing the powders and direct compression in a standard rotary tablet press. Tablet hardness is between 7 and 12 kP.

TABLE 1

| Component | Weight (mg) |
|---|---|
| Sodium alendronate monohydrate | 11.6 mg* |
| Hydroxypropyl methylcellulose | 25 mg |
| Lactose | 25 mg |
| Magnesium stearate | 0.5 mg |

*equivalent to 10 mg alendronic acid

Calcitriol, 0.05 mg, is dissolved in 20 ml of ethanol. Hydroxypropyl methylcellulose (HPMC), 136 g, is granulated with the ethanol solution for two minutes in a high shear mixer (e.g. Diosna). The granulate is dried at 40° C. and milled through a 0.63 mm sieve. The calcitriol granulate is then dry mixed with 400 g of hydroxypropyl cellulose (HPC), 80 g of tannic acid and 176 g croscarmellose sodium for five minutes. Magnesium stearate, 8 g, is then added and the mixture is mixed for another minute. The proportions of the blend are given in Table 2. The core is embedded in 800 mg of the blend by compression in a Kilian RUD-20 press coat machine. The outer tablet is of oval shape with dimensions about 17×7×9 mm.

TABLE 2

| Component | weight % |
|---|---|
| Calcitriol* | 6.25 × 10⁻⁶ |
| HPMC (Methocel K-15M) | 17 |
| Tannic acid | 10 |
| HPC (Klucel HF) | 50 |
| Crosscarmelose (aci-di-sol) | 22 |
| Magnesium stearate | 1 |

*Calcitriol is dosed at 0.05 μg per tablet

The resulting tablet provides immediate gastric release of calcitriol and delayed gastric release of alendronate after 2 h. Alendronate is released over about 4 h.

### Example 2

Sodium alendronate monohydrate is formulated into an immediate release core of 5-mm diameter with the composition of Table 3 by mixing the powders and direct compression in a standard rotary tablet press. Tablet hardness is between 7 and 12 kP.

TABLE 3

| Component | Weight (mg) |
|---|---|
| Sodium alendronate monohydrate | 11.6 mg* |
| Microcrystalline cellulose | 30 mg |
| Lactose for direct compression | 20 mg |
| Magnesium stearate | 0.5 mg |

*equivalent to 10 mg alendronic acid

Calcitriol is granulated and the gastric retention blend is prepared as described in Example 1. The core is embedded in 800 mg of the blend by compression in a Kilian RUD-20 press coat machine. The outer tablet is of oval shape with dimensions about 17×7×9 mm. The resulting tablet provides immediate gastric release of calcitriol and delayed gastric release of alendronate that begins after about 2 h. Alendronate is released over about 1 h.

### Example 3

A core containing monosodium alendronate monohydrate is prepared as described in Example 1. The core is embedded into 800 mg of the gastric retention composition of Table 4 formed by dry mixing of the components and compression in a Kilian RUD-20 press coat machine. The outer tablet is of oval shape with dimensions approximately 17×7×9 mm.

TABLE 4

| GRDS Component | weight % |
|---|---|
| HPMC (Methocel ® K-15M) | 17 |
| Tannic acid | 10 |
| HPC (Klucel ® HF) | 50 |
| Crosscarmelose (aci-di-sol ®) | 22 |
| Magnesium stearate | 1 |

Eight hundred grams of these tablets are coated by dissolving 25 g of HPC LF in 2 L of ethanol. Calcitriol, 0.05 mg, is dissolved in 20 ml of ethanol and added to the HPC solution. The solution is mixed for one minute. The tablets are spray coated in a perforated pan coater at a bed temperature of about 35° C. and air inlet temperature of 45° C. The tablets are air dried until the bed temperature reaches 45° C. The resulting tablets have a uniform coating containing 0.05 μg of calcitriol per tablet.

### Example 4

### In-Vivo Study of the Effect of Delivering Alendronate as a Combination Drug Regemin with Calcitrol

An in vivo study in an animal model was conducted to determine whether the novel combination drug regimen of calcitriol and alendronate improves the bioavailability of alendronate.

Six female beagle dogs, each approximately 2 years old and weighing approximately 9 kg were the animal models in this study. The same animals were used in each of two separate treatment sessions lasting 22-24 hours each. There was a 7 day wash-out period between sessions. The clinical state of each dog was checked within 48 hours prior to each treatment session and again after the last session. In each session the animals were dosed in the fasted state (n.p.o. 10-12 hours). The dogs were fed a standardized meal (200-250 g, Shur-Gain, Canada) four hours after dosing with alendronate.

During each session, the dogs were housed in steel metabolic cages. Urine samples were recovered from the bottom of the metabolic cages. At each collection point, a representative sample of urine (ca. 15 ml) was taken in a capped polypropylene vial and immediately frozen at −20° C. The remainder of the sample was frozen and retained.

Urine samples were analyzed for alendronate by high performance liquid chromatography (HPLC) (Anapharm, Canada).

In each session, the study drug was administered in the AM, in the fasted state, with 250 ml water (regulated at pH=2) administered via gastroesophogeal tube. During the monitoring (collection) period of each session, dogs were hydrated orally (syringe) every two hours with 200-250 ml water. As noted above, a meal was allowed 4 hours after the administration of alendronate.

In the first (reference) study session, alendronate (10 mg, Fosamax®) was administered in 250 ml pH regulated water via a gastroesophegeal tube.

In the second study session, a pre-dose of calcitrol (0.25 mg, Rocaltrol® was administered with 10-20 ml tap water. Two hours following the clacitrol pre-dose, alendronate (10 mg, Fosamax) was administered with 250 ml pH regulated water via gastroesophogeal tube.

Cumulative levels of alendronate in urine was determined at 0, 3, 6, 9, and 12 hours following alendronate dosage.

The results of the analyses of alendronate in urine for the two treatments are reported in Tables 5 and 6 and the average values are compared graphically in FIG. 1. In five of six dogs, the Fosamax→(Table 5) gave a total of ˜225-300 μg of alendronate in the urine over 12 hours. The sixth dog gave very low values but there were analytical problems with two of the urine samples, including the three hour sample which should have the highest values. The average value, without the data for dog #648, is 257.6 μg, and with all the data 225.9 μg, for a bioavalability of 2.6% or 2.3% respectively. These values are close to literature values in the dog. When the dogs were pretreated with calcitriol, followed by alendronate 2 hours later, the values of alendronate found in the urine were higher. The values ranged from 322 μg to 1016 μg. The average value was 527.5 μg. This value translates into a bioavailability of 5.3% which is twice as high as the value without the pretreatment. It is noted that the bioavailability was as high as 10% in one of the dogs.

TABLE 5
Alendronate Excreted in Dog Urine - Fosamax→

| | Fosamax - fasted | | | | | | |
|---|---|---|---|---|---|---|---|
| | | Alendronate (ug) | | | | | |
| | | excreted in Dog Urine | | | | | |
| | time (hr) | 0 | 3 | 6 | 9 | 12 | TOTAL |
| animal # | 295 | blq | 257.51 | 22.92 | 7.34 | 4.55 | 292.32 |
| | 109 | blq | 185.57 | 35.04 | 4.55 | 2.82 | 227.98 |
| | 612 | blq | 188.77 | 42.71 | 15.71 | 4.39 | 251.58 |
| | 648 | blq | nrv | 27.94 | 7.51 | nrv | 35.45 |
| | 005 | blq | 181.03 | 72.97 | 15.46 | nrv | 269.46 |
| | 578 | blq | 211.94 | 52.25 | 9.71 | 4.62 | 278.52 |
| | avg= | 0 | 205.0 | 42.3 | 10.0 | 4.1 | 225.9 |
| | avg | | 205.0 | 44.7 | 10.5 | 4.1 | 257.6 |
| | w/o648 | | | | | | |

blq = below level of quantitation
nrv = no reported value

TABLE 6
Alendronate Excreted in Dog Urine - Rocaltrol→ + Fosamax ™→

| | Rocaltrol + Fosamax - fasted | | | | | | |
|---|---|---|---|---|---|---|---|
| | | Alendronate (ug) | | | | | |
| | | excreted in Dog Urine | | | | | |
| | time (hr) | 0 | 3 | 6 | 9 | 12 | TOTAL |
| animal # | 295 | blq | 442.05 | 75.91 | 6.13 | 8.05 | 532.14 |
| | 109 | blq | 362.84 | 22.1 | 9.57 | 8.96 | 403.47 |
| | 612 | blq | 280.88 | nrv | 30.54 | 11.36 | 322.78 |
| | 648 | blq | 941.1 | nrv | 8.51 | 66.01 | 1015.62 |
| | 005 | blq | 407.82 | 41.15 | 5.94 | 10.12 | 465.03 |
| | 578 | 2.62 | 396.87 | 23.16 | nrv | 6.01 | 426.04 |
| | avg= | 2.6 | 471.9 | 40.6 | 12.1 | 18.4 | 527.5 |

### Example 5

### In-Vivo on Improving the Bioavailability of Alendronate: Effect of Varying Predose Intervals of Calcitriol in a Combination Drug Regimen with Alendronate

An in vivo study in an animal model was conducted to determine whether calcitrol, administered at varying predose intervals in combination therapy with alendronate increased the bioavailability of alendronate compared with the administration of alendronate alone.

six female beagle dogs, each approximately 2 years old and weighing approximately 9 kg were the animal models in this study. The same animals were used in each of five separate treatment sessions lasting 22-24 hours each, the duration of each session depending on the predose test interval being measured. The same drugs at identical dosages were administered in every treatment, viz., calcitriol (ROCALTROL®, 25 μg gel capsule; ROCHE) was the Vitamin D₃ analog administered as the predose drug and alendronate sodium (Fosamax®, 10 mg tablet, Merck, Sharp & Dohme) was the bis-phosphonate administered as the therapeutic drug. There was a 7 day wash-out period between sessions. The clinical state of each dog was checked within 48 hours prior to each treatment session and again after the last session. In each session the animals were dosed in the fasted state (n.p.o. 10-12 hours). The dogs were fed a standardized meal (Shur-Gain, Canada, 200-250 grams) four hours after administration of the therapeutic dose of alendronate.

During each session, the dogs were housed in steel metabolic cages. Urine samples were recovered from the bottom of the metabolic cages. At each collection point, two representative samples of urine (ca. 15 ml each) were taken in capped polypropylene vials and immediately frozen at −20° C. The remainder of the sample was frozen and retained.

Urine samples were analyzed for alendronate by HPLC with fluorescence detection (Anapharm, Inc., Quebec, Canada).

In each session, the predose study drug, calcitriol, was administered in the A.M., in the fasted state, with 10-20 ml tap water to facilitate swallowing, followed by hydration with 250 ml tap water (adjusted to pH=2.0) via gastroesophageal tube. During the monitoring (collection) period of each session, dogs were hydrated via gastroesophageal tube with 200-250 ml tap water (adjusted to pH=2.0) on the evening prior to initiation of each testing session and subsequently, with 200-250 ml pH-adjusted tap water every two hours post-administration of the therapeutic dose of alendronate, for up to 10 hours. As noted above, a meal was allowed 4 hours after the administration of alendronate.

In the first (reference) study session, the therapeutic dose of alendronate was administered alone, with hydration by administration of 250 ml pH-adjusted tap water via a gastroesophageal tube.

In the second (reference) study session, the predose of calcitriol and the therapeutic dose of alendronate were administered simultaneously, with 10-20 ml tap water to facilitate swallowing, immediately followed by 250 ml pH-adjusted tap water via a gastroesophageal tube.

In the third through sixth study sessions, the predose of calcitriol was administered with 10-20 ml tap water. At intervals of 1, 2, 3, or 6 hours, respectively, following the administration of the predose of calcitriol in each of the consecutive study sessions, the therapeutic dose of alendronate was administered with 10-20 ml tap water, immediately followed by 250 ml tap water via gastroesophageal tube.

For each calcitriol predose time interval tested, cumulative levels of alendronate concentrations in urine were determined over 12 hours post-administration of the therapeutic alendronate dose at collection time points beginning at the 0 hour prior to alendronate dose and again at 3, 6, 9, and 12 hours following the alendronate dose.

The results of the analyses of alendronate in urine for the five treatments are reported in Table 7. Table 7 collects the average of the total excreted alendronate as a function of the time interval between calcitriol administration and alendronate administration.

The results showed that the total alendronate bioavailability increased considerably 3 hours after the administration of calcitriol. Alendronate bioavailability without the vitamin D analog in this dog model was about 30 μg to 50 μg. Calcitriol, administered 3 hours before the alendronate administration increased this value to 108 μg. By delaying the release of the calcitriol predose for 3 to 5 hours and waiting for a time interval of several hours before administering the bis-phosphonate, the combination drug regimen is both more effective and more convenient.

TABLE 7
Average Total Alendronate Excreted as a Function of the Time
Interval Between Calcitriol and Alendronate Administrations

| Hours between administrations | total alendronate (μg) |
|---|---|
| 0 | 56.9 |
| 1 | 56.8 |
| 2 | 70.0 |
| 3 | 108.1 |
| 6 | 36.0 |

### Example 6

### In-Vivo Study on Improving the Bioavailability of Alendronate: Effect of Varying Predose Intervals of Alphacalcidol in a Combination Drug Regimen with Alendronate

An in vivo study in an animal model was conducted to determine whether alphacalcidol, administered in varying predose intervals in combination therapy with alendronate increased the bioavailability of alendronate compared with the administration of alendronate alone.

Six female beagle dogs, each approximately 2 years old and weighing approximately 9 kg were the animal models in this study. The same animals were used in each of five separate treatment sessions lasting 34-42 hours each, the duration of each session depending on the predose test interval being measured. The same drugs at identical dosages were administered in every treatment, viz., alphacalcidol (ALPHA D3®, 1.0 μg get capsule; TEVA) was the Vitamin D₃ analogue administered as the predose drug and alendronate sodium (Fosalan®, 10 mg tablet, Merck, Sharp & Dohme) was the bis-phosphonate administered as the therapeutic drug. There was a 7 day wash-out period between sessions. The clinical state of each dog was checked within 48 hours prior to each treatment session and again after the last session. In each session the animals were dosed in the fasted state (n.p.o. 10-12 hours). The dogs were fed a standardized meal (canned Bonzo meat, 1 full can, 425 grams) four hours after administration of the therapeutic dose of alendronate.

During each session, the dogs were housed in steel metabolic cages. Urine samples were recovered from the bottom of the metabolic cages. At each collection point, two representative samples of urine (ca. 5 ml each) were taken in capped polypropylene vials and immediately frozen at −20° C. The remainder of the sample was frozen and retained.

Urine samples were analyzed for alendronate by HPLC with fluorescence detection (Anapharm, Inc., Quebec, Canada).

In each session, the predose study drug, alphacalcidol, was administered in the A.M., in the fasted state, with 10-20 ml tap water to facilitate swallowing. During the monitoring (collection) period of each session, dogs were hydrated via gastroesophageal tube with 300 ml tap water on the evening prior to initiation of each testing session and subsequently, with 150 ml tap water every two hours post-administration of the therapeutic dose of alendronate, for up to 10 hours. As noted above, a meal was allowed 4 hours after the administration of alendronate.

In the first (reference) study session, the predose of alphacalcidol and the therapeutic dose of alendronate were administered simultaneously, with 10-20 ml tap water to facilitate swallowing, immediately followed by 250 ml tap water via a gastroesophageal tube.

In the second through fifth study sessions, the predose of alphacalcidol was administered with 10-20 ml tap water. At intervals of 1, 2, 3, or 6 hours, respectively, following the administration of the predose of alphacalcidol in each of the consecutive study sessions, the therapeutic dose of alendronate was administered with 10-20 ml tap water, immediately followed by 250 ml tap water via gastroesophageal tube.

For each alphacalcidol predose time interval tested, cumulative levels of alendronate concentrations in urine were determined over 24 hours post-administration of the therapeutic alendronate dose at collection time points beginning at the 0 hour prior to alendronate dose and again at 3, 6, 9, and 24 hours following the alendronate dose.

The results of the analyses of alendronate in urine for the five treatments are reported in Tables 8A-8E, 9 and 10. Tables 8A-8E give the results of the excretion of alendronate into dog urine for each of the experimental sessions. Table 9 collects the average of the total excreted alendronate as a function of the time interval between alphacalcidol administration and alendronate administration. Table 10 gives the average of total excreted alendronate as a function of the time interval between calcitriol administration and alendronate administration carried out in a separate experiment.

The results showed that the total alendronate bioavailability increased considerably 6 hours after the administration of alphacalcidol. It is expected that this increase will continue to be found when the time interval between administration of the predose of alphacalcidol and the subsequent administration of the therapeutic dose of alendronate is increased to 8, 10 or 12 hours. Alendronate bioavailability without the vitamin D analogue in this dog model was about 30 μg to 50 μg. Calcitriol, administered 3 hours before the alendronate administration increased this value to 108 μg. The improvement in alendronate bioavailability was similar for the two vitamin D analogues, calcitriol and alphacalcidol, but the optimal time interval between administration of the predose and maximum alendronate bioavailability was delayed in the case of alphacalcidol. This delay can be used to advantage in designing a combination drug regimen with a dose scheme that is convenient and improves the bioavailability of alendronate.

TABLE 8A
(alendronate 0 hours after 1-alpha)
SUMMARY OF ALENDRONATE QUANTITY
EXCRETED (μg) IN URINE

| Subject | Period | Draw Times (Hour) | | | | | |
|---|---|---|---|---|---|---|---|
| # | # | 0.000 | 3.00 | 6.00 | 9.00 | 24.0 | total |
| 295 | 1 | BLQ | NRV | 6.09 | NRV | NRV | 6.09 |
| 109 | 1 | BLQ | 27.33 | 4.27 | BLQ | 4.20 | 35.80 |
| 612 | 1 | BLQ | 28.01 | NRV | NRV | 2.91 | 30.92 |
| 648 | 1 | BLQ | 40.99 | 6.90 | 8.45 | 7.02 | 63.36 |
| 005 | 1 | BLQ | 28.26 | 8.87 | 3.52 | 2.90 | 43.55 |
| 578 | 1 | BLQ | 25.29 | 13.99 | 2.21 | 3.68 | 45.17 |
| | | | | | | avg = | 37.48 |

BLQ: Below Level of Quantitation
NRV: No Reportable Value

TABLE 8B
(alendronate 1 hour after 1-alpha)
SUMMARY OF ALENDRONATE QUANTITY
EXCRETED (μg) IN URINE

| | | |
|---|---|---|
| Subject | Period | Draw Times (Hour) |

| # | # | 0.000 | 3.00 | 6.00 | 9.00 | 24.0 | total |
|---|---|---|---|---|---|---|---|
| 295 | 2 | BLQ | 42.05 | 4.71 | NRV | 7.58 | 54.34 |
| 109 | 2 | BLQ | 39.99 | 4.01 | NRV | 3.47 | 47.47 |
| 612 | 2 | BLQ | 39.73 | 4.42 | 4.30 | 4.58 | 53.03 |
| 648 | 2 | 1.61 | 109.98 | 9.02 | 7.51 | 8.79 | 136.91 |
| 005 | 2 | BLQ | BLQ | BLQ | BLQ | BLQ | 0.00 |
| 578 | 2 | BLQ | 4.87 | 1.82 | NRV | NRV | 6.69 |
| | | | | | | avg = | 49.74 |

BLQ: Below Level of Quantitation
NRV: No Reportable Value

TABLE 8C
(alendronate 2 hours after 1-alpha)
SUMMARY OF ALENDRONATE QUANTITY
EXCRETED (μg) IN URINE

| Subject | Period | Draw Times (Hour) | | | | | |
|---|---|---|---|---|---|---|---|
| # | # | 0.000 | 3.00 | 6.00 | 9.00 | 24.0 | total |
| 295 | 3 | 3.14 | 25.52 | NRV | 3.82 | 5.84 | 38.32 |
| 109 | 3 | BLQ | NRV | NRV | 2.30 | 2.97 | 5.27 |
| 612 | 3 | BLQ | NRV | 4.11 | 2.73 | 4.11 | 10.95 |
| 648 | 3 | BLQ | 38.98 | 20.58 | 7.32 | 11.28 | 78.16 |
| 005 | 3 | NRV | BLQ | NRV | NRV | NRV | 0.00 |
| 578 | 3 | BLQ | 16.10 | 9.63 | 1.80 | NRV | 27.53 |
| | | | | | | avg = | 26.71 |

BLQ: Below Level of Quantitation
NRV: No Reportable Value

TABLE 8D
(alendronate 3 hours after 1-alpha)
SUMMARY OF ALENDRONATE QUANTITY
EXCRETED (μg) IN URINE

| Subject | Period | Draw Times (Hour) | | | | | |
|---|---|---|---|---|---|---|---|
| # | # | 0.000 | 3.00 | 6.00 | 9.00 | 24.0 | total |
| 295 | 4 | NRV | 59.51 | 5.68 | 3.87 | 3.97 | 73.03 |
| 109 | 4 | BLQ | 81.05 | 7.04 | NRV | 5.06 | 93.15 |
| 612 | 4 | BLQ | 35.52 | 5.01 | NRV | 4.12 | 44.65 |
| 648 | 4 | BLQ | 47.20 | 7.05 | 5.72 | NRV | 59.97 |
| 005 | 4 | BLQ | 51.07 | 11.11 | 13.24 | 20.07 | 95.49 |
| 578 | 4 | 3.81 | 85.63 | 13.41 | 7.10 | 6.81 | 116.76 |
| | | | | | | avg = | 80.51 |

BLQ: Below Level of Quantitation
NRV: No Reportable Value

TABLE 8E
(alendronate 6 hours after 1-alpha)
SUMMARY OF ALENDRONATE QUANTITY
EXCRETED (μg) IN URINE

| Subject | Period | Draw Times (Hour) | | | | | |
|---|---|---|---|---|---|---|---|
| # | # | 0.000 | 3.00 | 6.00 | 9.00 | 24.0 | total |
| 295 | 5 | BLQ | 65.02 | 31.98 | 6.03 | 7.28 | 110.31 |
| 109 | 5 | BLQ | 49.30 | 5.09 | 2.39 | 4.64 | 61.42 |
| 612 | 5 | 4.10 | 111.68 | 10.42 | 4.61 | 12.55 | 143.36 |
| 648 | 5 | NRV | 68.15 | 8.78 | 4.25 | 5.27 | 86.45 |
| 005 | 5 | 4.73 | 65.30 | 2.55 | 6.54 | 5.56 | 84.68 |
| 578 | 5 | NRV | 75.32 | 11.65 | 3.71 | 3.67 | 94.35 |
| | | | | | | avg = | 96.76 |

BLQ: Below Level of Quantitation
NRV: No Reportable Value

TABLE 9
Average Total Alendronate Excreted as a Function of the Time
Interval Between Alphacalcidol and Alendronate Administrations

| Hours between administrations | total alendronate (μg) |
|---|---|
| 0 | 37.5 |
| 1 | 49.7 |
| 2 | 26.7 |
| 3 | 80.5 |
| 6 | 96.8 |

TABLE 10
Average Total Alendronate Excreted as a Function of the Time
Interval Between Calcitriol and Alendronate Administrations

| Hours between administrations | total alendronate (μg) |
|---|---|
| 0 | 56.9 |
| 1 | 56.8 |
| 2 | 70.0 |
| 3 | 108.1 |
| 6 | 36.0 |

### Example 7

### In-Vivo Study on Improving the Bioavailability of Alendronate: Effect of Varying Predoses in a Combination Drug Regimen with Alendronate

An in vivo study in an animal model was conducted to determine whether calcitriol or alphacalcidol, administered in varying predose intervals in combination therapy with alendronate increased the bioavailability of alendronate compared with the administration of alendronate alone.

The method of example 6 was used. This study compared sessions of Fosalen alone, predosing of alphacalcidol at predose intervals of 6 hours, 8 hours, and 10 hours, and predosing with calcitriol at a predose interval of 3 hours. The results are shown below in Table 11.

TABLE 11
Cumulative Alendronate in Urine (ugm)

| | | calcitriol | alphacalcidol | alphacalcidol | alphacalcidol |
|---|---|---|---|---|---|
| | fosalen | 3 hr | 6 hr | 8 hr | 10 hr |
| dog # | alone | predose | predose | predose | predose |
| 205 | 48.84 | 106.68 | 77.78 | 110.49 | 233.93 |
| 109 | 65.52 | 73.68 | 44.34 | 90.85 | 48.66 |
| 612 | 20.67 | 51.48 | 81.69 | 116.53 | 86.74 |
| 648 | 83.88 | 138.12 | 77.83 | 95.71 | 192.41 |
| 005 | 34.06 | 136.86 | 104.74 | 85.29 | 64.46 |
| 578 | 61.75 | 222.4 | 69.57 | 77.67 | 12.23 |
| avg | 52.5 | 121.5 | 76.0 | 96.1 | 106.4 |
| median | 55.3 | 121.8 | 77.8 | 93.3 | 75.6 |
| std dev | 22.8 | 60.2 | 19.5 | 14.9 | 87.2 |

Having thus described the invention with reference to various preferred embodiments, those skilled in the art will appreciate modifications of these exemplary embodiments that do not depart from the spirit and scope of the invention as defined by the claims that follow.

1. A method of increasing the bioavailability of a bis-phosphonate comprising administering an effective predose of a vitamin D derivative, and after a time interval, administering a therapeutic dose of a bis-phosphonate, wherein the bis-phosphonate is selected from the group consisting of alendronate, risedronate, etidronate, zoledronate, and tiludronate.

2. A method of increasing the bioavailability of a bis-phosphonate comprising administering an effective predose of alphacalcidol, and after a time interval, administering a therapeutic dose of a bis-phosphonate.

3. A method of increasing the bioavailability of a bis-phosphonate comprising administering an effective predose of calcitriol, and after a time interval, administering a therapeutic dose of a bis-phosphonate.

4. A method of increasing the bioavailability of a bis-phosphonate comprising administering an effective predose of a vitamin D derivative, and after a time interval, administering a therapeutic dose of a bis-phosphonate, wherein the time interval is about equal to the amount of time required for blood calcium level to reach a maximum after administering the vitamin D derivative.

5. The method of claim 4, wherein the vitamin D derivative is calcitriol and the time interval is about 3 hours to about 5 hours.

6. The method of claim 4, wherein the vitamin D derivative is alphacalcidol and the time interval is about 6 hours to about 14 hours.

7. A method of increasing the bioavailability of a bis-phosphonate comprising administering an effective predose of a vitamin D derivative, and after a time interval, administering a therapeutic dose of a bis-phosphonate, wherein the time interval is at least 6 hours and the bis-phosphonate is selected from the group consisting of alendronate, risedronate, etidronate, zoledronate, and tiludronate.

8. The method of claim 7, wherein the vitamin D derivative is selected from the group consisting of calcitriol, alphacalcidol, 24,25-dihydroxy vitamin D₃, and calcifediol.

9. The method of claim 8, wherein the vitamin D derivative is alphacalcidol.

10. The method of claim 9, wherein the predose of alphacalcidol is about 0.2 μg to about 2 μg.

11. The method of claim 7, wherein the bis-phosphonate is alendronate.

12. The method of claim 11, wherein the dose of alendronate is about 10 mg to about 70 mg.

13. The method of claim 7, wherein the time interval is about 6 hours to about 14 hours.

14. The method of claim 13, wherein the time interval is about 6 hours to about 12 hours.

15. The method of claim 14, wherein the time interval is about 6 hours to about 10 hours.

16. The method of claim 7, wherein the predose of vitamin D derivative is administered at bedtime and the dose of bis-phosphonate is administered before eating.

17. The method of claim 7, wherein the time interval is a period of fasting.

18. A method of increasing the bioavailability of a bis-phosphonate comprising administering a delayed-release effective predose of vitamin D derivative, and after a time interval, administering a therapeutic dose of a bis-phosphonate wherein the bis-phosphonate is selected from the group consisting of alendronate, risedronate, etidronate, zoledronate, and tiludronate.

19. The method of claim 18, wherein the vitamin D derivative is selected from the group consisting of calcitriol, alphacalcidol, 24,25-dihydroxy vitamin D₃, and calcifediol.

20. The method of claim 19, wherein the vitamin D derivative is calcitriol.

21. The method of claim 20, wherein the predose of calcitriol is about 0.2 μg to about 2 μg.

22. The method of claim 18, wherein the release of the predose of vitamin D derivative is delayed about 3 hours to about 5 hours.

23. The method of claim 22, wherein the release of the predose of vitamin D derivative is delayed about 3 hours.

24. The method of claim 18, wherein the delayed-release predose of vitamin D derivative is a dosage form with a delayed-release enteric coating.

25. The method of claim 18, wherein the bis-phosphonate is alendronate.

26. The method of claim 25, wherein the dose of alendronate is about 10 mg to about 70 mg.

27. The method of claim 18, wherein the time interval is at least 6 hours.

28. The method of claim 27, wherein the time interval is about 6 hours to about 14 hours.

29. The method of claim 28, wherein the time interval is about 6 hours to about 12 hours.

30. The method of claim 29, wherein the time interval is about 6 hours to about 10 hours.

31. The method of claim 18, wherein the predose of vitamin D derivative is administered at bedtime, and the dose of bis-phosphonate is administered before eating.

32. The method of claim 19, wherein the time interval is a period of fasting.
